(19) United States
(12) Patent Application Publication
(10) Pub. No.: US 2017/0028626 A1
(43) Pub. Date: Feb. 2, 2017

(54) Compact Drop-on-Demand Apparatus Using Light Actuation Through Optical Fibers

(71) Applicant: ECOLE POLYTECHNIQUE FEDERALE DE LAUSANNE (EPFL), Lausanne (CH)

(72) Inventors: Paul Delrot, Saint-Sulpice (CH); Demetri Psaltis, Preverenges (CH); Christophe Moser, Lausanne (CH)

(21) Appl. No.: 15/220,507

(22) Filed: Jul. 27, 2016

(30) Foreign Application Priority Data
Jul. 29, 2015 (IB) PCT/IB2015/055717

(51) Int. Cl. B29C 67/00; A61N 5/06; B33Y 30/00; A61C 19/06; A61M 31/00; B33Y 10/00; F21V 8/00; A61F 9/00

(52) U.S. Cl. CPC B29C 67/0059; G02B 6/0096; A61N 5/062; A61F 9/0026; A61C 19/063; A61M 31/00; B33Y 10/00; B33Y 30/00; A61N 2005/063; A61N 2005/067

(57) ABSTRACT

A drop delivery system comprising a light source; an optical waveguide bringing light from the light source; and a liquid supplying means configured to bring a liquid at a tip of the optical waveguide, wherein the light source and the optical waveguide are configured to enable the light to eject a drop of the liquid.

### CROSS REFERENCE TO RELATED APPLICATIONS

The present application claims foreign priority to the PCT filing with the Serial No. PCT/IB2015/055717, filed on Jul. 29, 2015, the entire contents thereof herewith incorporated by reference.

### FIELD OF THE INVENTION

The present invention relates to compact drop-on-demand systems, in particular, those systems that involve the absorption of light to eject micro-droplets of viscous or inviscid fluids.

### DISCUSSION OF THE BACKGROUND ART

Drop-on-demand technology is now a mature technology, having a profound impact on manufacturing processes, and is used for instance in inkjet printing, additive manufacturing or contactless drug delivery devices. Drop-on-demand systems can be classified according to the way they are actuated, their compactness, resolution, and the type of liquids they can dispense.

Most consumer desktop inkjet printers are based on thermal actuation. In each chamber, a resistor is in contact with the ink and upon actuation; it vaporizes a small volume of the ink, pushing towards the nozzle the rest of the ink, therefore generating a droplet. This approach allows for densely packing ink chambers and nozzles. The droplet size and therefore the resolution of the system are defined by the nozzle diameter. Furthermore to avoid clogging of the nozzle, the ink needs to have a low viscosity, typically around 10 mPa·s, and the nozzle diameter needs to be large enough, typically more than 50 μm. Murphy S V, Atala A., “3D bioprinting of tissues and organs”, Nat. Biotechnol 2014, vol. 32, pp. 773-785.

Another approach for compact drop-on-demand systems is the piezoelectric actuation, which also allows for the design of compact systems with liquids having similar rheological properties to the ones dispensed by thermal actuation. Piezoelectric-based dispensing systems are based on the generation of a pressure wave by the piezoelectric crystal contained in each chamber, hence pushing outwards a droplet. The drop diameter is also defined by the nozzle diameter. However, it has been shown that sub-nozzle resolution can be achieved by defining a proper cycle of positive and negative pressure waves and tuning the viscosity of the ink. See Chen A U, Basaran O A. “A new method for significantly reducing drop radius without reducing nozzle radius in drop-on-demand drop production” Phys Fluids, 2002, vol. 14, pp. L1-L4.

Several approaches attempted to use light-actuation for drop-on-demand systems, for instance using light to drive the isomerization of surfactants from cis to trans, it was demonstrated that it is possible to dispense droplets on demand by tuning the surface tension of a liquid in suspension. See for example Shin J Y, Abbott N L. “Using light to control dynamic surface tensions of aqueous solutions of water soluble surfactants” Langmuir 1999, vol. 15, pp. 4404-10. The main drawbacks of this method are that the droplet size is only defined by gravity and surface tension, and that the method is limited to specific liquids. Another approach consists in focusing a femtosecond laser pulse on a micrometric spot close to the open surface of a liquid reservoir. Even for weakly absorbing liquids, the tight focusing allows to overcome the optical breakdown threshold, leading to the generation of a transient bubble close to the surface and the generation of a jet. See Duocastella M, Patrascioiu A, Fernández-Pradas J M, Morenza J L, Serra P. “Film-free laser forward printing of transparent and weakly absorbing liquids” Opt. Express, 2010, vol. 18, pp. 21815-25.

The most mature light-assisted drop-on-demand technology is perhaps Laser-assisted Forward Transfer (LIFT) See U.S. Pat. No. 7,014,885. In LIFT, large light transparent ribbons (typically more than 20 mm) are coated with a thin (20 nm-30 nm) solid-state film of metal, such as gold, which is light absorbing. The ribbons are then covered with a thin layer of ink, typically a few tens of microns thick, and set upside down, on top of the receiving substrate. An infrared or ultraviolet nanosecond laser pulse is then focused on a spot on the top of the ribbon. When a sufficient energy is transferred to the light-absorbing film, this film vaporizes the small volume of ink directly in contact with the metal layer that was illuminated. When the laser energy is high enough, the bubble expansion allows for the ejection of at least one droplet towards the substrate. The absence of walls on the ribbon permits to dispense micrometric droplets of highly viscous fluids (1-300 mPa·s). See Guillemot F, Souquet A, Catros S, Guillotin B, Lopez J, Faucon M, et al. “High-throughput laser printing of cells and biomaterials for tissue engineering” Acta Biomater 2010; vol. 6, pp. 2494-2500. It has been demonstrated that the vaporization of the metallic light-absorbing layer results in the contamination of the droplets. This problem can be solved by using a thin micrometric layer of solid-state polymer instead of metal. With proper laser energy, the polymeric layer will generate a blister and dispense droplets. See Brown M S, Kattamis N T, Arnold C B, “Time-resolved study of polyimide absorption layers for blister-actuated laser-induced forward transfer,” Journal of Applied Physics, 2010, vol. 107, p. 3103. However, in LIFT the large ribbons used as ink cartridges makes it impossible for the system to be compact.

More recently, it has been demonstrated that a flow-focusing effect can take place when a light-triggered shockwave impinges on a concave meniscus at the open end of a water-filled micro-capillary. See Tagawa Y, Oudalov N, Visser C W, Peters I R, van der Meer D, Sun C, et al. “Highly Focused Supersonic Microjets,” Phys. Rev. X 2012, vol. 2, p. 031002. High velocity jets and micro-droplets were consequently produced on demand. Furthermore, as a proof of principle, this method was implemented as contactless drug-delivery system. See Tagawa Y, Oudalov N, Ghalbzouri A E, Sun C, Lohse D. “Needle-free injection into skin and soft matter with highly focused microjets,” Lab Chip 2013, vol. 13, pp. 1357-1363. However, this system also lacked of compactness as the laser pulses were delivered by a microscope objective aside of the liquid-filled micro-capillary.

Therefore, despite all the advancements in the fields of micro-droplet generation, ink-jet systems, and drop-on-demand solutions, further improvements are desired to address the drawbacks and issues of the available background art systems that are discussed above.

### SUMMARY

According to one aspect of the present invention, a device or system is provided that includes a structure and physical mechanism to generate micro-droplets from a liquid-filled delivery system by light-actuation. Preferably, this compact drop-on-demand system allows for liquids with a viscosity between 0.5 mPa·s and 200 mPa·s, but not limited to, to be ejected on demand as single micrometric droplets. The system first includes a micro-delivery system, such as a glass micro-capillary, but not limited to. In at least one embodiment, the inner walls of the system's nozzle are covered with a thin solid-state light-absorbing film. In at least one embodiment, the solid-state light-absorbing film is a metal. In at least one embodiment, an optical fiber or a bundle of optical fibers is enclosed in the delivery system so that a fluid can still flow in the system. The delivery system is filled with a liquid so that the liquid's meniscus is in proximity to the nozzle of the system and, in at least one embodiment part of the liquid is in contact with the light-absorbing film. In at least one embodiment of this invention, the liquid is light absorbing. A pulse of laser light is propagated through a waveguide and a spot or several spots of light are focused onto the light-absorbing film or the liquid. When the energy delivered into a laser spot is high enough, a transient bubble and a shockwave are generated. When the distance between the bubble and the liquid's meniscus is short enough, a flow-focusing effect takes place on the meniscus and one or several droplets of the liquid are ejected along the axis of the nozzle. The meniscus goes back to its initial shape shortly after the droplet breakup and allows for the generation of additional droplets. The solid-state light-absorbing film can be, but not limited to gold or platinum. In at least one embodiment of this invention, the delivery system is a capillary waveguide. Preferably, the laser pulse width can be, but not limited to 5 ns-2 μs. Preferably, the laser pulse energy can be, but not limited to 20 μJ-300 μJ. Preferably, the thickness of the light-absorbing layer can be, but not limited to 10 nm-10 μm. Preferably, the diameter of the capillary can be, but not limited to 10 μm-500 μm. In at least one embodiment, the solid-state light-absorbing film is a polymer, such as Kapton™ polyimide, but not limited to.

Furthermore, in at least one embodiment, an endoscopic drug delivery system is provided that can be actuated by light and the liquid that is discharged can have, but not limited to a biological or drug content. In at least one embodiment, a system and method for printing an article by light-actuation is provided, and the liquid to be discharged can contain, but not limited to, polymers, monomers, solvents and photo-initiators. Preferably, in this embodiment, three-dimensional structures can be printed.

An application example is for delivering a specific amount of drug at a specific location in the eye. According with yet another aspect of the present invention, a method for delivering a drug to a specific location in a body is provided, by using an endoscopic drug delivery system. Such specific location could be at the retina to treat eye diseases, or the brain or teeth or the ear such as in the cochlear. In a further embodiment, the endoscopic delivery system can deliver a polymerizable material to form, for example, but not limited to, a scaffold structure.

The summary of the invention is neither intended nor should it be construed as being representative of the full extent and the scope of the invention, which additional aspects will become more readily apparent from the detailed description, particularly when taken together with the appended drawings.

### BRIEF DESCRIPTION OF THE SEVERAL VIEWS OF THE DRAWINGS

The accompanying drawings, which are incorporated herein and constitute part of this specification, illustrate the presently preferred embodiments of the invention, and together with the general description given above and the detailed description given below, serve to explain features of the invention.

FIG. 1 depicts an embodiment of the system before light-actuation. FIG. 1 is a cross-sectional drawing of an embodiment of the compact drop-on-demand system showing an optical waveguide enclosed in a liquid-filled capillary. Reference numeral 100 corresponds to the liquid to dispense, reference numeral 101 is the meniscus of the liquid contained in a micro-capillary 102, reference numeral 103 is an optical fiber enclosed in the delivery system and reference numeral 104 is a thin solid-state light-absorbing film.

FIG. 2 depicts an embodiment of the system after light-actuation. FIG. 2 is a cross-sectional drawing of the delivery system showing an optical waveguide 200 enclosed in a micro-capillary 201 whose nozzle is covered with a solid-state light-absorbing film 202. A light pulse 203 is focused onto a spot onto the film 202, hence vaporizing part of the liquid 204 and leading to the dispensing of a micro-droplet 205. Reference numeral 206 corresponds to the transient bubble generated by the laser pulse, and reference numeral 207 corresponds to the liquid's meniscus.

FIG. 3 shows an embodiment of the system with a light-absorbing liquid and multi-bubble generation. FIG. 3 is another embodiment of the compact drop-on-demand system with a light-absorbing liquid. Reference numeral 300 corresponds to an optical waveguide enclosed in a micro-capillary 301 filled with the liquid to jet 302. A pulse of light propagates in the waveguide and is focused onto several spots 303 in the liquid, generating several bubbles 304 and a micro-droplet 305. Reference numeral 306 corresponds to the liquid's meniscus.

FIG. 4 shows an embodiment of the system with a capillary optical waveguide. FIG. 4 is another embodiment of the compact drop-on-demand system comprising a liquid 400, filled capillary optical waveguide 401, whose nozzle is covered with a thin solid-state light-absorbing film 402. Reference numeral 403 corresponds to the liquid's meniscus. Reference numeral 404 corresponds to a micro-droplet generated by the transient-bubble 405 generation. This bubble is generated by guiding a pulse of light 406 through the capillary waveguide and focusing it onto the light-absorbing film.

FIG. 5 depicts an embodiment of the system with a tapered nozzle. FIG. 5 is another embodiment of the delivery system using a tapered micro-capillary 500 in which an optical waveguide 501 is enclosed. Reference numeral 502 corresponds to a solid-state thin light-absorbing film, reference numeral 503 to the transient bubble generated by focusing a pulse of light 504 through the waveguide onto the light-absorbing film. Reference numeral 505 corresponds to the liquid to dispense, reference numeral 506 to the liquid's meniscus and 507 to the dispensed micro-droplet of liquid.

FIG. 6 shows an embodiment of the system with a single-mode fiber and a light-absorbing layer deposited on the tip of the fiber. FIG. 6 is another embodiment of the delivery system in which a single-mode fiber 600 is enclosed in a micro-capillary 601 filled with a liquid 602. The light is absorbed by a thin light-absorbing layer 603 deposited on the distal tip of the fiber creates a bubble 604 which leads to the generation of a droplet 605. The distance 606 between the single-mode fiber and the meniscus 607 can be changed in order to create a bubble at a different location.

FIG. 7 shows an embodiment of the system with a capillary optical waveguide and the printing of a three-dimensional (3D) structure. FIG. 7 is another embodiment of the compact drop-on-demand system comprising a liquid 700, filled capillary optical waveguide 701, whose nozzle is covered with a thin solid-state light-absorbing film 702. Reference numeral 703 corresponds to the liquid's meniscus. Reference numeral 704 corresponds to a three-dimensional structure on top of a receiving substrate 705 built by successive ejections of droplets generated by transient-bubbles 706 generation. These successive bubbles are generated by guiding a pulse of light 707 through the capillary waveguide and focusing it onto the light-absorbing film. The structure is cured by guiding light 708 to each droplet through the capillary waveguide.

FIG. 8 shows an embodiment of the system with a capillary optical waveguide and an imaging system to control the delivery of the droplets. FIG. 8 shows another embodiment where a set of droplets 800 is printed on a substrate 801 using a liquid-filled 802 capillary optical waveguide 803. The printed structure is imaged with an imaging system formed by an optical fiber 804 by collecting light 805 emitted by a light source 806.

FIGS. 9A to 9D depict images of the generation of droplets with fluids of different viscosities. FIG. 9A shows MMA, FIG. 9B shows HDDA, FIG. 9C shows TMPTA, and FIG. 9D shows SU-8 with 50% of solid content. FIGS. 9A to 9D show a compilation of experimental results obtained with a 425-μm wide capillary. FIG. 9A is the generation of a single droplet of MMA, reference numeral 900 is the meniscus of MMA, reference numeral 901 the capillary inner walls, and reference numeral 902 the generated droplet. FIG. 9B is the generation of a single droplet of HDDA, reference numeral 903 is the meniscus of HDDA, reference numeral 904 the generated droplet, and reference numeral 905 the capillary inner walls. FIG. 9C is the generation and deposition of a single droplet of TMPTA, reference numeral 906 is the generated droplet, reference numeral 907 the reflection of the droplet on the receiving substrate, and reference numeral 908 a previously deposited droplet. FIG. 9D is the generation and deposition of a single droplet of Su-8 with 50% of solid content, reference numeral 909 is the forming droplet of SU-8 followed by a thin thread of SU-8 910 that broke up from the meniscus, reference numeral 911 is the reflection of the pendant droplet on the receiving substrate and reference numeral 912 a previously deposited droplet.

FIGS. 10A and 10B represent the dependency of the jetting regimes of the experimental drop-on-demand device on the laser pulse energy for inks of various viscosities: FIG. 10A shows the Newtonian water-glycerol mixtures, and FIG. 10B non-Newtonian polymer inks.

FIG. 11 shows the dependency of the droplet diameter on the Ohnesorge number of Newtonian water-glycerol mixtures for various capillary diameters.

FIGS. 12A and 12B depict a pattern printed with the drop-on-demand device and HDDA on a glass substrate. FIG. 12A shows the HDDA droplets and FIG. 12B the extracted contours of the droplets and the targeted position of each droplet. FIGS. 12A and 12B depict an example of a pattern printed with the experimental drop-on-demand device. Reference numeral 1200 is a droplet of HDDA deposited on a glass substrate, reference numeral 1201 is the targeted position of the droplet shown with 1200, and reference numeral 1202 is the extracted contour of one of the droplets.

FIGS. 13A and 13B show patterns of columns alternatively printed with mouse and rabbit immunoglobulins, with FIG. 13A showing patterns imaged under brightfield microscopy before an immunoassay, and FIG. 13B showing patterns under fluorescence imaging after an immunoassay. FIGS. 13A and 13B show an example of a pattern printed with biological content, in FIG. 13B the droplets surrounded by the white rectangles exhibit the fluorescence of the specific marker for rabbit immunoglobulin whereas the remaining droplets exhibit the fluorescence of the specific marker for mouse immunoglobulin.

Herein, identical reference numerals are used, where possible, to designate identical elements that are common to the figures. Also, the images in the drawings are simplified for illustration purposes and may not be depicted to scale.

### DETAILED DESCRIPTION OF THE PREFERRED EMBODIMENTS

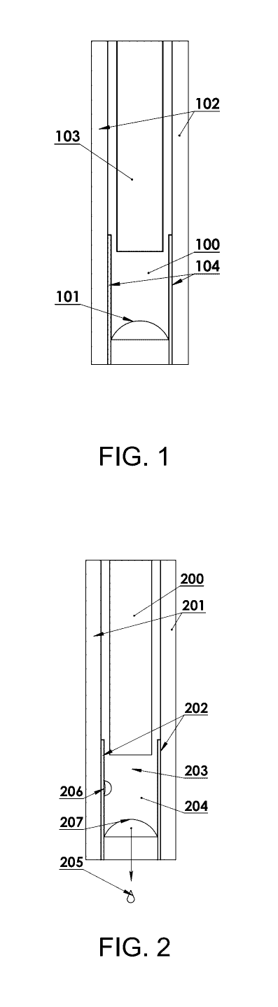

The techniques, apparatus, materials and systems as described in this specification can be used to implement a compact light-actuated drop-on-demand system. Described is a compact light-actuated liquid dispensing device and system composed of a delivery system, and at least one optical waveguide. FIGS. 1 and 2 show a depiction of a cross-section of an embodiment of the system, in which an optical waveguide, such as a multimode optical fiber, is enclosed a capillary filled with the liquid to dispense. With a proper wetting behavior of the liquid, a concave meniscus is formed at the liquid-air interface.

According to another aspect of the present invention, an actuation is proposed with focused spots of light through optical fibers, overcoming several drawbacks of the background solutions. The propose actuation method allows for a compact, light-actuated drop-on-demand system with a sub-nozzle resolution. The device that employs this method is also able to dispense highly viscous liquids. With this method, system, and device, a digital phase conjugation technique can be used for generating a sharp focus point at an end of a multimode optical fiber, for the modulation of optical wavefronts. See Papadopoulos I, Farahi S, Moser C, Psaltis D., “Focusing and scanning light through a multimode optical fiber using digital phase conjugation,” Opt. Express, 2012, vol. 20, pp. 10583-10590.

FIG. 2 shows a laser light pulse propagating in the optical waveguide and focused, using for instance a digital phase conjugation technique, onto a thin solid-state light-absorbing film. The light-absorbing film can be, but not limited to, made of metal or polymer, and deposited by, for instance, electroless plating and deep-coating respectively. When the light pulse energy is high enough, the heat generated on the light-absorbing film suddenly vaporizes a small volume of liquid, hence generating a bubble and a shockwave. When the bubble is generated in sufficient proximity to the liquid's meniscus, for instance a capillary's diameter away from the meniscus, the shockwave impact on the fluid's meniscus results in a flow-focusing effect, hence creating a thin jet. If the kinetic energy of the tip of this thin jet is large enough to overcome surface tension and the viscoelastic drag of the fluid, at least one droplet is released along the axis of the micro-capillary. Because of the flow-focusing effect, the droplet has a diameter significantly smaller than the nozzle of the delivery system.

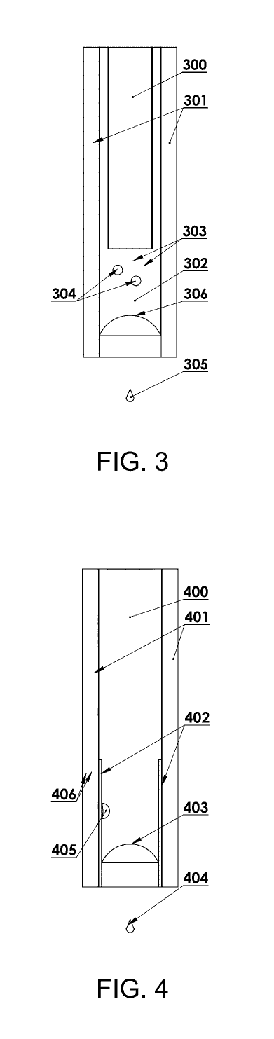

In at least one embodiment of the present invention, the device can be operated with a light-absorbing liquid as described in FIG. 3. Furthermore, multiple spots can be focused at the output of the optical waveguide, generating several bubbles and shockwaves.

In at least one embodiment of the invention, the device can be operated with a capillary optical waveguide as described in FIG. 4, which allows for an even more compact delivery system as no optical waveguide needs to be enclosed in the delivery system.

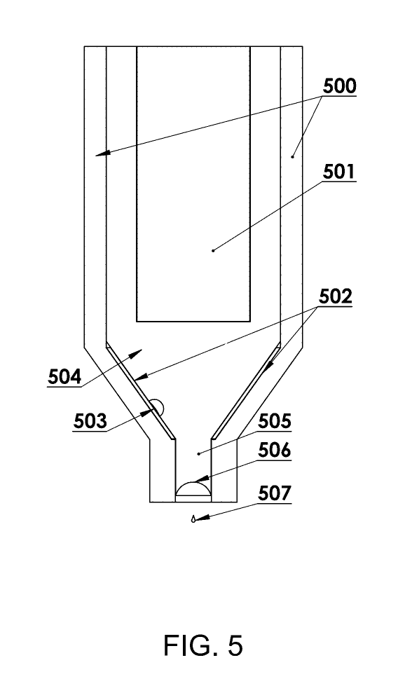

Furthermore, in at least one embodiment of the device, the delivery system can have, but not limited to, a tapered nozzle as described in FIG. 5, which allows for a larger number of optical waveguides to be enclosed in the delivery system while maintaining a small droplet size. The droplet size is indeed decreasing with the diameter of the part of the nozzle in which the liquid's meniscus is located.

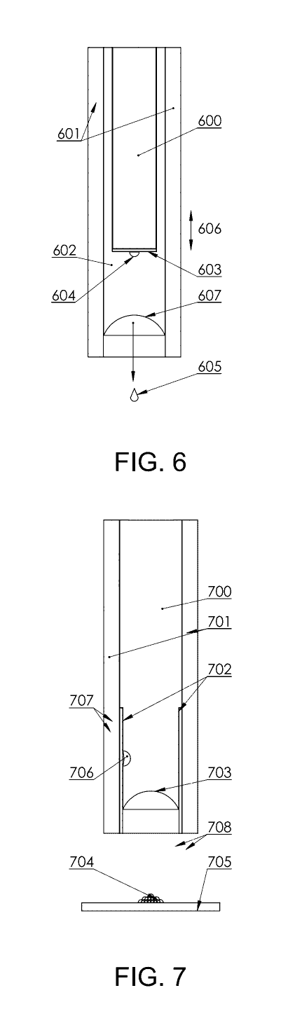

In at least one embodiment of the invention, the device can be operated with a single-mode fiber as a means to create a bubble on the distal tip of the fiber and consequently generate one or more droplets as described in FIG. 6. As in a single-mode fiber the transverse intensity is described by a Gaussian function; a bubble can be generated at the center of the fiber's distal tip. Moreover, in at least one embodiment of the invention, the drop-on-demand device can be used to print an article by successive polymerization of the generated droplets as described in FIG. 7. In this embodiment, the ink can be, but not limited to, a photo-polymer whose curing can be, but not limited to, achieved by bringing light through the delivery system as described in FIG. 7. This allows to perform a method of printing three-dimensional structures.

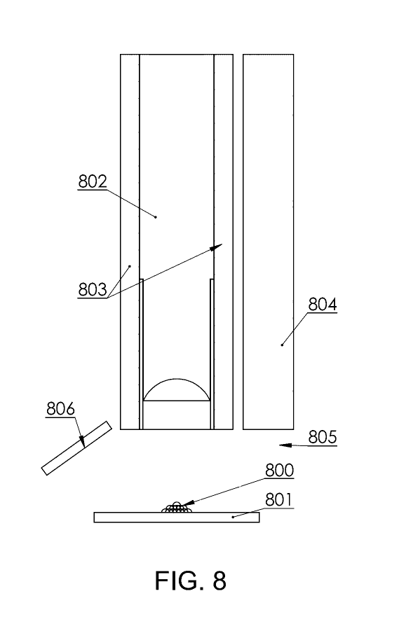

Lastly, in at least one embodiment of the invention, the device can operate with an imaging system alongside the delivery system in order to control the delivery of the droplets. The imaging device can operate, but not limited to, via an optical fiber collecting the light emitted by a source, as described in FIG. 8 and bringing it to a sensor.

Next, a proof of principle demonstration and measurements for the system and device are presented. As a proof of principle glass micro-capillaries of inner diameter ranging from 100 μm to 420 μm were filled with a non-Newtonian polymeric ink, such as SU-8, methyl methacrylate (MMA), 1,6-hexanediol diacrylate (HDDA), and trimethylpropane triacrylate (TMPTA). The inks were stained with an organic dye having a peak absorption in the green part of the spectrum of light. Furthermore, the inks had solid contents between 0% and 50%, which resulted in viscosities ranging from 0.6 mPa·s to 150 mPa·s. Moreover, due to surface tension, the inks had a contact angle with the micro-capillary's glass interface between 30° and 55°. Stained Newtonian water-glycerol mixtures of viscosities ranging from 2 mPa·s to 210 mPa·s were also used. The Newtonian inks we had a contact angle with the micro-capillary's glass interface between 25° and 35°. By focusing a green laser pulse, with a temporal width of 5 ns, and energy between 3 μJ and 70 μJ, on a spot located in the ink contained in the capillary and close to the walls of the capillaries, a small volume of the ink was vaporized, hence generating a transient bubble. When the distance between the bubble and the meniscus formed by the ink at the open end of the capillary was short enough, for example of the order of a few hundreds of microns, the shockwave generated by the bubble allowed for a flow-focusing effect at the meniscus interface, thereby generating one or more micro-droplets. The results demonstrate that the method can generate single micro-droplet on demand for fluids with a viscosity ranging from 0.6 mPa·s to 148±11 mPa·s for non-Newtonian inks and from 2 mPa·s to 210 mPa·s for the Newtonian inks. The results also demonstrate that the diameter of the single droplet increases with the viscosity of the ink but still remains small compared to the capillary's diameter. The achieved sub-nozzle resolution shows that clogging would therefore not impede the proposed system. Moreover, the velocity of the produced micro-droplets was measured between 0.5 m/s and 5 m/s, the velocity increasing with the energy sent to generate the bubble.

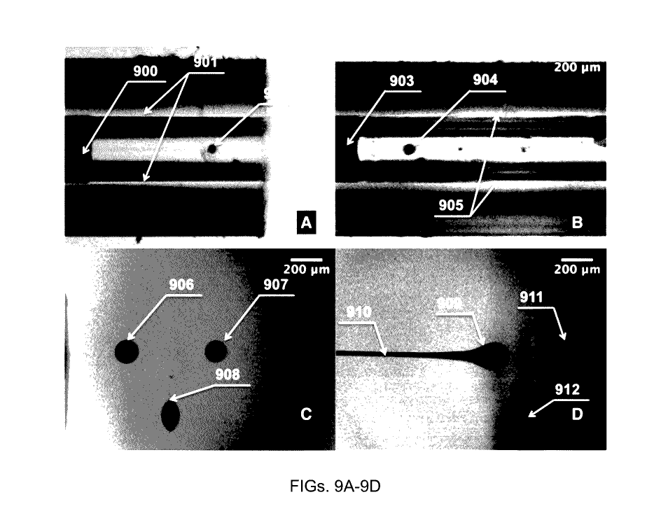

FIGS. 9A to 9D represent experimental results of a set of droplet generation on demand from a micro-capillary of inner-diameter 300 μm as mentioned above and for different inks of increasing viscosities. The diameter of the droplets is obviously small compared to the micro-capillary's diameter, which allows for printing with a sub-nozzle resolution.

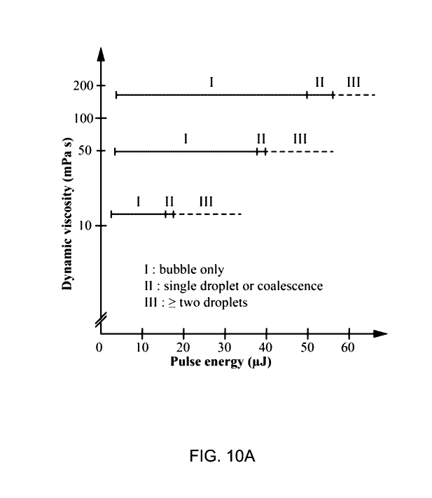

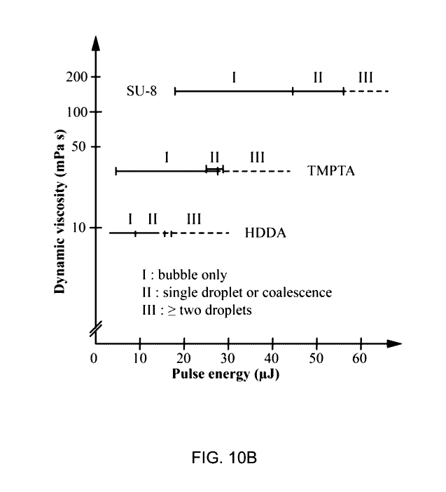

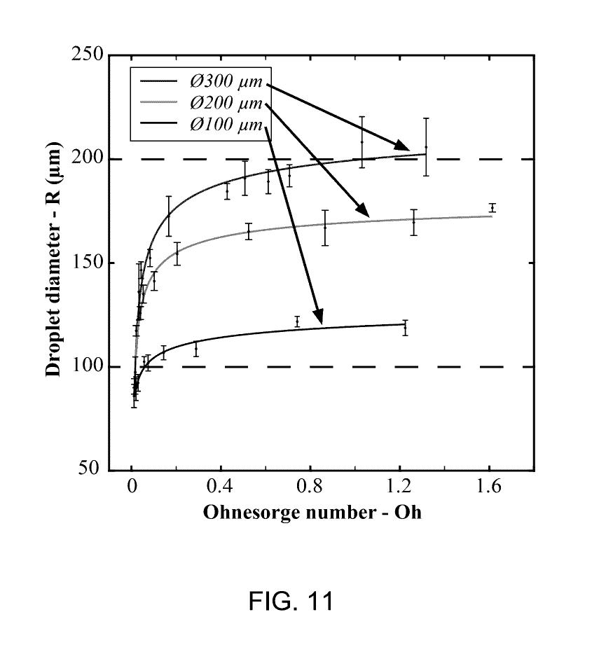

FIGS. 10A and 10B show the jetting regimes measured with the set up described above for a capillary diameter of 300 μm. For Newtonian and non-Newtonian inks of various viscosity; the laser pulse energy can be adapted to achieve satellite-free droplet generation; thus ensuring a clean printing. FIG. 11 shows the measured relationship between the diameter of a single droplet generated with a given ink on the Ohnesorge number of the said droplet where the Ohnesorge number Oh of the droplet is Oh=η/\sqrt{L*σ*ρ} with η the dynamic viscosity of the ink, L the size of the droplet, τ the surface tension of the ink and ρ the density of the ink. Some analysis showed that single droplet generation is only possible for 0.01<Oh<1 as at low Oh number, satellite droplets would be generated alongside the main droplet and at high Oh number, viscous damping would not allow for the ejection of the ink. See Reis N, Derby B. 2000, “Ink jet deposition of ceramic suspensions: modelling and experiments of droplet formation.” MRS Symp. Proc., Vol. 624, pp. 65-70. However, our results demonstrate successful generation of droplets for highly viscous inks (η=148±11 mPa·s, Oh=1.36±0.13) with our device.

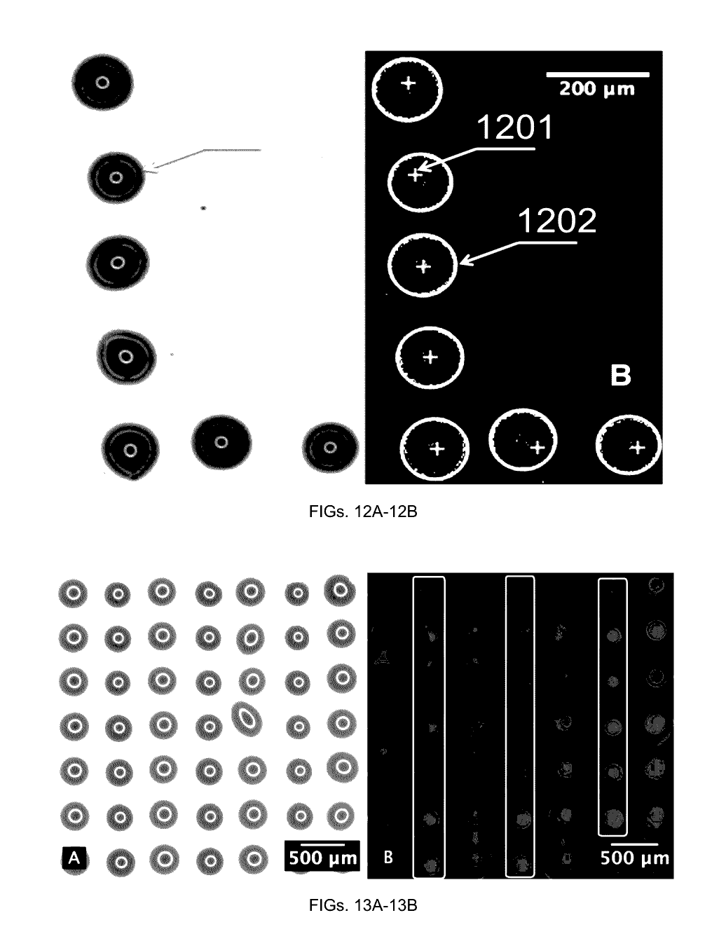

Lastly, to demonstrate that the current method can be used to print structures, a flat glass substrate was placed 2 mm below the distal tip of the capillary and a right-angle pattern, shown in FIGS. 12A and 12B, was created by moving the substrate between each droplet ejection. As can be seen, the drop-on-demand system showed good reproducibility and under stable operation, the accuracy of the system was calculated to be ±13 μm. Furthermore, the potential of the current method for printing drugs was demonstrated. Columns of mouse and rabbit immunoglobulins were alternatively printed on a flat glass substrate and imaged under brightfield microscopy prior to an immunoassay as shown in FIG. 13A. After which, an immunoassay was performed and imaged under fluorescent microscopy, shown in FIG. 13B. The fluorescent imaging demonstrated that the functionality of these biologically relevant samples was not affected by the printing process since the fluorescent markers were still selectively bound to the printed immunoglobulins.

The design and fabrication of a drop-on-demand apparatus using light actuation through optical fibers have been described. By focusing a spot of laser light through an optical waveguide, such as a multimode optical fiber enclosed in the delivery system, onto a thin light-absorbing layer, a bubble and a shockwave are generated and allow for the ejection of at least one micro-droplet of the liquid. Moreover, this compact delivery system allows for the generation of micro-droplet of fluids with high viscosities. Furthermore, depending on the rheological properties of the liquid, the droplet diameter can range down to one order of magnitude smaller than the capillary's diameter.

While the invention has been disclosed with reference to certain preferred embodiments, numerous modifications, alterations, and changes to the described embodiments are possible without departing from the sphere and scope of the invention, as defined in the appended claims and their equivalents thereof. Accordingly, it is intended that the invention not be limited to the described embodiments, but that it have the full scope defined by the language of the following claims.

1. A drop delivery system comprising
a light source;
an optical waveguide bringing light from the light source; and
a liquid supplying device configured to bring a liquid at a tip of the optical waveguide,
wherein the light source and the optical waveguide are configured to enable the light to eject a drop of the liquid.

2. The system of claim 1, wherein the optical waveguide is a single mode waveguide.

3. The system of claim 2, wherein the single mode waveguide is an optical fiber.

4. The system of claim 1, wherein the optical waveguide is a multimode waveguide.

5. The system of claim 4, wherein the multimode waveguide is an optical fiber.

6. The system of claim 5, wherein the optical fiber includes at least one of a step index fiber, gradient index fiber, multi-core fiber, and tapered fiber.

7. The system of claim 1, wherein the optical waveguide is a bundle of multimode or single-mode optical fibers.

8. The system of claim 7, wherein the multimode optical fibers include at least one of step index fibers, gradient index fibers, multi-core fibers, and tapered fibers.

9. The system of claim 1, wherein the light source is configured to provide the light as a pulse of light.

10. The system of claim 1, further comprising:
a nozzle having inner walls,
wherein the inner walls of the nozzle are covered with a thin light-absorbing film of metal or polymer.

11. The system of claim 1, further comprising:
a nozzle,
wherein the optical waveguide is enclosed in the drop delivery system to guide light to the nozzle, and allowing for liquid flows in the system.

12. The system of claim 1, wherein
the optical waveguide is enclosed in a transparent capillary, a distal tip of the transparent capillary being covered with a thin solid-state light-absorbing film of metal or polymer, and
the system further comprises a device to bring a thin layer of the liquid in contact with an open surface of the thin solid-state light-absorbing film.

13. The system of claim 1, wherein the drop delivery system is a capillary optical waveguide.

14. The system of claim 13, where the capillary optical waveguide is arranged to bring light onto an ejected drop in order to polymerize the ejected drop.

15. A method for printing an article with a drop delivery system, the drop delivery system including,
a light source, an optical waveguide bringing light from the light source, and a liquid supplying device configured to bring a liquid at a tip of the optical waveguide, the light source and the optical waveguide are configured to enable the light to eject a drop of the liquid,
the method including a step of ejecting the drop of the liquid onto the article to be printed.

16. The method for claim 15, wherein the article includes at least one of transparent optical structures, biological structures, prostheses, and electrical structures.

17. A method for endoscopic drug delivery with a drop delivery system, the drop delivery system including,
a light source, an optical waveguide bringing light from the light source, and a liquid supplying device configured to bring a liquid at a tip of the optical waveguide, the light source and the optical waveguide are configured to enable the light to eject a drop of the liquid,
the method including a step of ejecting the drop of the liquid onto a desired location in a body.

18. The method of claim 17, wherein the location in the body includes at least one of an eye, a cochlear, a tooth, or a brain.

19. The system of claim 1, further comprising:
an imaging system arranged alongside the system and configured to image the delivery of drops.

20. The system of claim 19, wherein the imaging system is configured to bring light on deposited droplets in order to polymerize the deposited droplets.
